(19) United States
(12) Patent Application Publication
(10) Pub. No.: US 2024/0279261 A1
(43) Pub. Date: Aug. 22, 2024

(54) METHODS FOR PREPARING HIGH-PURITY ARABINOSE CRYSTALS

(71) Applicant: ZHEJIANG HUAKANG PHARMACEUTICAL CO., LTD., Quzhou (CN)

(72) Inventors: Xinfeng HAN, Quzhou (CN); Dongxu LI, Quzhou (CN); Mingqian YANG, Quzhou (CN); Chengjun LIAO, Quzhou (CN); Shufang QIN, Quzhou (CN); Jiaxing LUO, Quzhou (CN)

(73) Assignee: ZHEJIANG HUAKANG PHARMACEUTICAL CO., LTD., Quzhou (CN)

(21) Appl. No.: 18/651,633

(22) Filed: Apr. 30, 2024

Related U.S. Application Data
(63) Continuation of application No. PCT/CN2023/096370, filed on May 25, 2023.

(30) Foreign Application Priority Data
Dec. 17, 2022 (CN) 202211628377.2

(51) Int. Cl. C07H 3/02; B01D 1/28; B01D 9/00; B01D 15/36; B01D 21/26; B01D 69/02; B01F 21/00; B01F 35/21; C07H 1/06

(52) U.S. Cl. CPC C07H 3/02; B01D 1/28; B01D 9/0022; B01D 9/0031; B01D 15/361; B01D 21/262; B01D 69/02; B01F 21/00; B01F 35/2115; C07H 1/06; B01D 2009/0086; B01D 2325/02834

(57) ABSTRACT

Embodiments of the present disclosure provide a method for preparing a high-purity arabinose crystal, comprising operations of dissolving, blending, ion exchange, decolorization and filtration, fine filtration, evaporation and concentration, crystallization, centrifugation, and drying performed in sequence on a low-purity arabinose crystal. Throughout the operations of preparing the arabinose crystal, a pH value of a material may be controlled between 4.3 and 7.5, and a temperature may not exceed 70° C. This prevents arabinose from transforming into impurities under a high temperature, thereby maintaining and improving a purity of the prepared arabinose crystal.

### CROSS-REFERENCE TO RELATED APPLICATIONS

This application is a continuation of International Patent Application No. PCT/CN2023/096370, filed on May 25, 2023, which claims priority to Chinese Patent Application No. 202211628377.2, filed on Dec. 17, 2022, the entire contents of each of which are hereby incorporated by reference.

### TECHNICAL FIELD

The present disclosure relates to the field of sugar alcohol preparation technology, and in particular to a method for preparing a high-purity arabinose crystal.

### BACKGROUND

Arabinose is widely used in fields such as pharmaceuticals and health foods. A low-purity arabinose crystal product is inexpensive but fails to meet customer demands. On the other hand, a high-purity arabinose crystal product, due to its complex processes and production difficulties, commands higher prices but can meet specific customer requirements for process formulations, thereby possessing a significant market space.

In an existing production process, L-arabinose, due to its characteristics, may be transformed into other substances by reaction under a high temperature and a low pH value. For example, the L-arabinose may be transformed into xylitol by isomerization. As another example, the L-arabinose may break down into small molecules below five carbons by decomposition. As yet another example, the L-arabinose may transform into maltotriose by polymerization. Consequently, the arabinose content in the L-arabinose decreases continuously in the production process, particularly during operations like decolorization, evaporation, and chromatographic separation, making it challenging to obtain a high-purity arabinose crystal with purity above 99.5%.

CN112079886A discloses a method for improving the purity of xylitol and arabinose through chromatographic separation. However, in the production process such as decolorization and chromatographic separation, the transformation rate of arabinose is low due to a relatively high temperature and a relatively low pH value. Therefore, it is desirable to provide a method for preparing a high-purity arabinose crystal.

### SUMMARY

One or more embodiments of the present disclosure provide a method for preparing a high-purity arabinose crystal. The method may include:

Operation 1, dissolution: dissolving a low-purity arabinose crystal to obtain a dissolved sugar solution using a dissolution tank system, wherein the dissolution tank system may be provided with a temperature sensor and an automatic regulating valve, a Siemens distributed control system (DCS) may be used to monitor a temperature during the dissolution to ensure that a temperature of the dissolved sugar solution is in a range of 55° C.-60° C., and an arabinose content in the low-purity arabinose crystal is 96%-97%.

Operation 2, blending: directing the dissolved sugar solution through a pipeline into a blending tank, adding an arabinose centrifugation mother liquor from operation 8 of a previous round of operations to the blending tank, and obtaining a blended sugar solution by blending the dissolved sugar solution with the arabinose centrifugation mother liquor, wherein a pH value of the blended sugar solution may be in a range of 4.3-5.0, a dry basis concentration may be in a range of 50%-60%, and the arabinose content of the blended sugar solution may be 94±0.5%.

Operation 3, ion exchange: performing an ion exchange operation on the blended sugar solution to obtain an ion-exchanged sugar solution, wherein a temperature of the ion-exchanged sugar solution may be controlled at 45° C.-50° C. through a heat exchanger for ion exchange feed, and a pH value of the ion-exchanged sugar solution may be in a range of 5.5-6.5.

Operation 4, decolorization and filtration: directing the ion-exchanged sugar solution through a pipeline into a decolorization tank for decolorization and filtration to obtain a decolorized sugar solution, wherein a decolorization temperature for decolorization and filtration may be controlled at 60° C.-65° C., and a pH value for decolorization and filtration may be in a range of 5.5-7.5.

Operation 5, fine filtration: performing a fine filtration operation on the decolorized sugar solution using a fine filtration membrane with an aperture of 0.45 μm to obtain a refined sugar solution, wherein a temperature for fine filtration may be controlled at 50° C.-65° C., and a pH value for fine filtration may be in a range of 5.0-7.5.

Operation 6, evaporation and concentration: directing the refined sugar solution into a mechanical vapor recompression (MVR) evaporator for evaporation and concentration to obtain a concentrated sugar solution, wherein a temperature for evaporation and concentration may be at 65° C.-70° C., and a pH value for evaporation and concentration may be in a range of 5.0-7.5.

Operation 7, crystallization: introducing the concentrated sugar solution into a vacuum sugar-cooking system for crystallization, wherein a temperature for crystallization may be at 63° C.-65° C., and a vacuum level for crystallization may be in a range of 70 mbar-90 mbar.

Operation 8, centrifugation: performing a centrifugal separation operation on a material obtained in operation 7 to separate a solid arabinose and an arabinose centrifugation mother liquor from the material, wherein the arabinose centrifugation mother liquor may be directed through the pipeline into the blending tank for blending in operation 2 and an arabinose content in the arabinose centrifugation mother liquor may be in a range of 89%-91%.

Operation 9, drying: performing a drying operation on the solid arabinose with 80° C. hot air to obtain the high-purity arabinose crystal, wherein an arabinose content may be greater than 99.8%.

In some embodiments, in operation 3, an ion exchange column may be used in the ion exchange operation, and a ratio of cationic resins to anionic resins in the ion exchange column may be 7:10.

In some embodiments, in operation 4, the decolorization and filtration operation may include adding activated carbon to the decolorization tank at a ratio of 1.2-1.4 Kg/ton of the dry basis, stirring at 110 rpm for 30-45 minutes for decolorization, and using a plate and frame filter to filter and remove the activated carbon.

In some embodiments, in operation 7, the crystallization operation may include performing a vacuum evaporation crystallization by adding arabinose crystal seeds of 300-400 mesh in a ratio of two per ten thousand of the dry basis when an oversaturation degree of the concentrated sugar solution reaches 1.01-1.02, a crystallization period may be 8 hours, and a stirring speed may be at 80 rpm.

In some embodiments, in operation 8, the centrifugal separation operation may further include controlling a washing time at 10 seconds and maintaining a water temperature at 55° C.-60° C.

In some embodiments, in operation 9, the drying operation may further include controlling a moisture content at 0.15%-0.3%, and after the moisture content is in the range of 0.15%-0.3%, cooling the high-purity arabinose crystal to 20° C.-24° C. using clean cold air with a temperature in a range of 12° C.-15° C.

The method strictly controls the temperatures during the preparation operations, ensuring that the temperatures do not exceed 70° C., thereby preventing arabinose from transforming into impurities under a high temperature and reducing purity.

The method for preparing the high-purity arabinose crystal in the present disclosure has the following beneficial effects:

(1) The blending operation introduced before refinement ensures the stability of the liquid material.

(2) By adjusting the ratio of cationic resins to anionic resins to 7:10, the pH value of the ion-exchanged output (i.e., the ion-exchanged sugar solution) is stabilized at 5.5-6.5. This avoids local overalkalinization of the liquid material during pH adjustment by adding alkalis, preventing isomerization issue and maintaining production efficiency and product quality in subsequent operations.

(3) Using the low-purity arabinose crystal with a relatively higher impurity content, the method performs dissolution, purification, and concentration operations on the low-purity arabinose crystal to obtain the arabinose crystal with a purity greater than 99.8%. This results in a crystallization yield improvement of more than 3.5% compared to existing production processes.

### BRIEF DESCRIPTION OF THE DRAWINGS

The present disclosure is further illustrated by way of exemplary embodiments, which will be detailed with reference to the drawings. These embodiments are not restrictive. In these embodiments, the same numbers denote the same structures, wherein:

FIG. 1 is a flowchart illustrating an exemplary process for preparing a high-purity arabinose crystal according to some embodiments of the present disclosure.

### DETAILED DESCRIPTION

In order to more clearly illustrate the technical solutions and beneficial effects of the embodiments of the present disclosure, the accompanying drawings for the description of the embodiments are described below. It should be understood that the specific embodiments described herein are only intended to explain the present disclosure and are not intended to limit the scope of the present disclosure.

Some embodiments of the present disclosure provide a method for preparing a high-purity arabinose crystal. By controlling a pH value and a temperature during preparation to prevent isomerization, decomposition, and polymerization, the purity of the arabinose crystal can be enhanced.

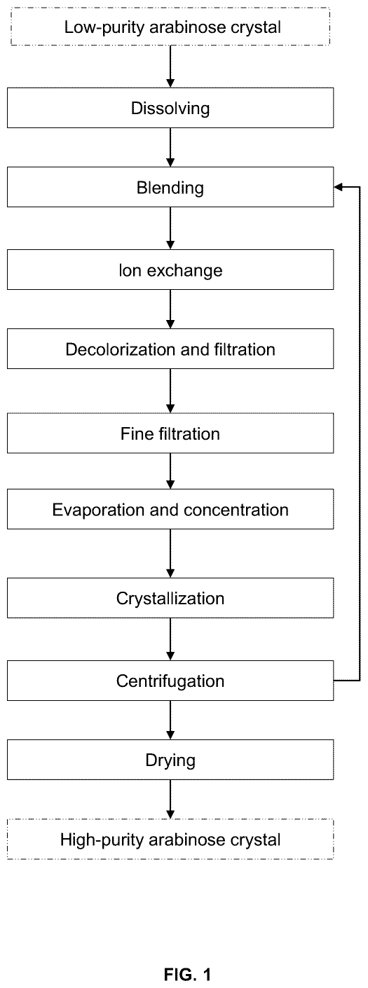

FIG. 1 is a flowchart illustrating an exemplary process for preparing a high-purity arabinose crystal according to some embodiments of the present disclosure. The directional arrows in the drawing indicate the flow direction of materials or the sequence of the operations. In some embodiments, operation 2-operation 9 may be performed multiple rounds until a condition is satisfied. For example, the condition may include that the arabinose content in the arabinose centrifugation mother liquor is smaller than a threshold, e.g., 89%, 90%, etc. The method may include:

Operation 1, dissolution. In operation 1, a low-purity arabinose crystal may be dissolved to obtain a dissolved sugar solution using a dissolution tank system. The dissolution tank system may be provided with a temperature sensor and an automatic regulating valve, and a Siemens distributed control system (DCS) may be used to monitor a temperature during the dissolution to ensure that a temperature of the dissolved sugar solution is in a range of 55° C.-60° C., thereby ensuring a stability of the dissolved sugar solution.

Arabinose is a type of levorotatory monosaccharide extracted through complex chemical and physical techniques from colloids secreted by Arabian trees. Arabinose has promising applications in fields such as weight loss, diabetes control, etc.

The temperature sensor is capable of sensing a temperature inside a dissolution tank of the dissolution tank system and converting the temperature into an output signal, which is then transmitted to the automatic regulating valve.

The automatic regulating valve may function as a variable resistance in a pipeline of the dissolution tank system, regulating parameters such as pressure, flow rate, temperature, etc., through automatic control. In some embodiments, the automatic regulating valve may receive a temperature signal transmitted by the temperature sensor and automatically adjusts the temperature inside the dissolution tank to prevent transformation of the arabinose crystal into other impurities under a condition of an excessively high temperature.

In some embodiments, the temperature of the dissolved sugar solution may be between 55° C. to 60° C.

In some embodiments, the temperature of the dissolved sugar solution may be 55° C., or 56° C., or 57° C., or 58° C., or 59° C., or 60° C., etc.

In some embodiments, a ratio of the mass of arabinose to the total mass of the low-purity arabinose crystal may be in a range of 96% to 97%.

In some embodiments, the ratio of the mass of arabinose to the total mass of the low-purity arabinose crystal may be 96%, or 96.5%, or 97%, etc.

Operation 2, blending. In operation 2, the dissolved sugar solution may be directed into a blending tank through a pipeline using an electromagnetic flow meter, an automatic regulating valve, and a Siemens intelligent control system, an arabinose centrifugation mother liquor from operation 8 of a previous round of operations may be added to the blending tank, and a blended sugar solution may be obtained by blending the dissolved sugar solution with the arabinose centrifugation mother liquor. The pH value of the blended sugar solution may be in a range of 4.3-5.0, a dry basis concentration may be in a range of 50%-60%, and the arabinose content of the blended sugar solution may be 94±0.5%.

The dry basis concentration of the blended sugar solution refers to a proportion of the absolute dry components in the blended sugar solution relative to the mass of the blended sugar solution. The dry basis concentration of the blended sugar solution may be expressed by the formula: dry basis concentration of blended sugar solution (%)=dry basis (mass of absolute dry components)/mass of corresponding blended sugar solution*100%.

In some embodiments, the dry basis concentration may range from 50% to 60%.

In some embodiments, the dry basis concentration may be 50%, or 52%, or 54%, or 56%, or 58%, or 60%, etc.

Operation 3, ion exchange. In operation 3, an ion exchange operation may be performed on the blended sugar solution to obtain an ion-exchanged sugar solution.

The temperature of the ion-exchanged sugar solution may be controlled at 45° C.-50° C. through a heat exchanger for ion exchange feed, and the pH value of the ion-exchanged sugar solution may be in a range of 5.5-6.5.

In some embodiments, the temperature of the ion-exchanged sugar solution may range from 45° C. to 50° C.

In some embodiments, the temperature of the ion-exchanged sugar solution may be 45° C., or 46° C., or 47° C., or 48° C., or 49° C., or 50° C., etc.

In some embodiments, the pH value of the ion-exchanged sugar solution may range from 5.5 to 6.5.

In some embodiments, the pH value of the ion-exchanged sugar solution may be 5.5, or 5.7, or 5.9, or 6.0, or 6.3, or 6.5, etc.

In some embodiments, an ion exchange column may be used in the ion exchange operation, a ratio of cationic resins to anionic resins in the ion exchange column may be 7:10, and the pH value of the ion-exchanged sugar solution may be maintained in the range of 5.5-6.5, thereby preventing the arabinose from being transformed into impurities under an excessively low pH value.

Operation 4, decolorization and filtration. In operation 4, the ion-exchanged sugar solution may be directed through a pipeline into a decolorization tank for decolorization and filtration to obtain a decolorized sugar solution.

In some embodiments, the decolorization temperature may range from 60° C. to 65° C.

In some embodiments, the decolorization temperature may be 60° C., 61° C., 62° C., 63° C., 64° C., 65° C., etc.

In some embodiments, the pH value of the decolorized sugar solution may range from 5.5 to 7.5.

In some embodiments, the pH value of the decolorized sugar solution may be 5.5, or 5.7, or 6.0, or 6.3, or 6.5, or 7.0, or 7.5, etc.

In some embodiments, in the decolorization filtration operation, activated carbon may be added to the decolorization tank at a ratio of 1.2-1.4 Kg/ton of the dry basis and stirred at 110 rpm for 30-45 minutes for decolorization, and a plate and frame filter may be used to filter and remove the activated carbon.

Operation 5, fine filtration. In operation 5, a fine filtration operation may be performed on the decolorized sugar solution using a fine filtration membrane with an aperture of 0.45 μm to obtain a refined sugar solution.

In some embodiments, the temperature for fine filtration may be controlled at 50° C.-65° C.

In some embodiments, the temperature for fine filtration may be 50° C., or 55° C., or 60° C., or 65° C., etc.

In some embodiments, the pH value for fine filtration may be in a range of 5.0-7.5.

In some embodiments, the pH value for fine filtration may be 5.0, or 5.5, or 6.0, or 6.5, or 7.0, or 7.5, etc.

Operation 6, evaporation and concentration. In operation 6, the refined sugar solution may be directed into a mechanical vapor recompression (MVR) evaporator for evaporation and concentration to obtain a concentrated sugar solution.

In some embodiments, the temperature for evaporation and concentration may be in a range of 65° C.-70° C.

In some embodiments, the temperature for evaporation and concentration may be 65° C., or 66° C., or 67° C., or 68° C., or 69° C., or 70° C., etc.

In some embodiments, the pH value for evaporation and concentration may be in a range of 5.0-7.5.

In some embodiments, the pH value for evaporation and concentration may be 5.0, or 5.5, or 6.0, or 6.5, or 7.0, or 7.5, etc.

Operation 7, crystallization. In operation 7, the concentrated sugar solution may be introduced into a vacuum sugar-cooking system for crystallization. The temperature for crystallization may be in a range of 63° C.-65° C., and the vacuum level for crystallization may be in a range of 70 mbar-90 mbar.

In some embodiments, the crystallization operation may include adding arabinose crystal seeds of 300-400 mesh in a ratio of two per ten thousand of the dry basis when the oversaturation degree of the concentrated sugar solution reaches 1.01-1.02. A crystallization period may be 8 hours, and a stirring speed may be at 80 rpm.

Operation 8, centrifugation. In operation 8, a centrifugal separation operation may be performed on a material obtained in operation 7 to separate a solid arabinose and an arabinose centrifugation mother liquor from the material. The arabinose centrifugation mother liquor may be directed through the pipeline into the blending tank for blending in operation 2 and an arabinose content in the arabinose centrifugation mother liquor may be in a range of 89%-91%.

In some embodiments, in the centrifugal separation operation, a washing time may be controlled at 10 seconds and a water temperature may be maintained at 55° C.-60° C.

In some embodiments, the water temperature for washing may be 55° C., or 56° C., or 57° C., or 58° C., or 59° C., or 60° C., etc.

Operation 9, drying. In operation 9, a drying operation may be performed on the solid arabinose with 80° C. hot air to obtain the high-purity arabinose crystal, and an arabinose content of the high-purity arabinose crystal is greater than 99.8%.

In some embodiments, in the drying operation, a moisture content may be controlled in a range of 0.15%-0.3%, and after the moisture content is in the range of 0.15%-0.3%, the high-purity arabinose crystal may be cooled to 20° C.-24° C. using clean cold air with temperature in a range of 12° C.-15° C.

The temperature during the preparation operations of the high-purity arabinose crystal is controlled to not exceed 70° C., thereby preventing arabinose from being transformed into impurities under a high temperature and reducing purity.

The method for preparing the high-purity arabinose crystal in the present disclosure is further illustrated through the following embodiments.

### Embodiments

The following embodiments provide further specific illustrations related to the method for preparing the high-purity arabinose crystal in the present disclosure. Some contents in the following embodiments may also be replaced or combined with corresponding contents from other embodiments to form new embodiments. Unless otherwise specified, the experimental techniques used in the following embodiments are conventional techniques. The test materials used in the following embodiments, unless otherwise specified, are purchased from standard biochemical reagent companies. Quantitative experiments in the following embodiments are conducted with three repetitions, and the results are averaged. It should be understood that the following embodiments are provided for better explanation of the present disclosure and are not intended to limit the scope of the disclosure.

### Embodiment 1: Determining an Impact of Temperature and pH Value on Arabinose Content

Embodiment 1 includes the following operations:

100 g of L-arabinose sample was taken and dissolved in water to form an arabinose solution with a refractive index of 60%. The pH value of the arabinose solution was adjusted to 2.7 and 4.3. The arabinose solution was sequentially heated at 60° C., 65° C., and 70° C. for 48 hours, respectively. Samples were collected at 24 hours and 48 hours for analysis and results are shown in Table 1.

TABLE 1
Components of the arabinose solution under different temperatures and pH values

| | | Content (%) | | | | | | | | | | | |
|---|---|---|---|---|---|---|---|---|---|---|---|---|---|
| | | pH2.7 | | | | | | pH4.3 | | | | | |
| | | 60° C. | | 65° C. | | 70° C. | | 60° C. | | 65° C. | | 70° C. | |
| Components | Initial | 24 h | 48 h | 24 h | 48 h | 24 h | 48 h | 24 h | 48 h | 24 h | 48 h | 24 h | 48 h |
| Arabinose | 98.34 | 97.6 | 96.75 | 97.33 | 95.96 | 96.83 | 95.4 | 98.3 | 98. 11 | 97.97 | 97.89 | 98.06 | 97.54 |
| Xylose | 1.36 | 1.55 | 1.78 | 1.88 | 2.09 | 1.9 | 2.23 | 1.42 | 1.61 | 1.71 | 1.75 | 1.68 | 1.84 |
| Maltotriose | 0.05 | 0.32 | 0.55 | 0.4 | 0.93 | 0.6 | 1.17 | 0.08 | 0.15 | 0.2 | 0.23 | 0.26 | 0.32 |
| Glucose/ | 0 | 0.12 | 0.29 | 0.11 | 0.19 | 0.11 | 0.13 | 0 | 0 | 0 | 0.06 | 0 | 0.08 |
| Sorbitol | | | | | | | | | | | | | |

Comparing the results, as the pH value decreases and the heating temperature increases during arabinose production, the arabinose content decreases more rapidly over time. The pH value is identified as a main factor causing a decline in the arabinose content. When the pH value is at 4.3 or above, and the material is heated at 75° C. for 48 hours, the arabinose content decreases only by 2.31%. However, when the pH value is at 2.7, and the material is heated at 75° C. for 48 hours, the arabinose content decreases by 8.9%, which is more than three times the decrease of the arabinose content when the pH value is at 4.3.

The results indicate that effectively adjusting the pH value of the arabinose solution to above 4.3 can reduce the impact of high temperature on the arabinose content, avoiding to lower the processing temperature for content control, thereby maintaining production efficiency.

### Embodiment 2

One embodiment of the method for preparing the high-purity arabinose crystal in the present disclosure includes:

Operation 1, dissolution: a purchased low-purity arabinose crystal was dissolved to obtain a dissolved sugar solution using a dissolution tank system. The dissolution tank system was provided with a temperature sensor and an automatic regulating valve, a Siemens DCS was used to monitor a temperature during the dissolution to ensure that a temperature of the dissolved sugar solution was in a range of 55° C.-60° C., and the arabinose content in the low-purity arabinose crystal was 96%.

Operation 2, blending: the dissolved sugar solution obtained in operation 1 was directed through a pipeline into a blending tank using an electromagnetic flowmeter, an automatic regulating valve, and a Siemens intelligent control system. An arabinose centrifugation mother liquor from operation 8 of a previous round of operations was added to the blending tank, and a blended sugar solution was obtained by blending the dissolved sugar solution with the arabinose centrifugation mother liquor. The pH value of the blended sugar solution was 5.0, a dry basis concentration was 50%, and the arabinose content of the blended sugar solution was 93.84%.

Operation 3, ion exchange: an ion exchange operation was performed on the blended sugar solution obtained in operation 2 to obtain an ion-exchanged sugar solution using an ion exchange system. The temperature of the ion-exchanged sugar solution was controlled at 45° C.-50° C. through a heat exchanger for ion exchange feed, a ratio of cationic resins to anionic resins of the ion exchange system was adjusted to be 7:10, and the pH value of the ion-exchanged sugar solution was 6.5.

Operation 4, decolorization and filtration: the ion-exchanged sugar solution was directed through a pipeline into a decolorization tank, activated carbon was added to the decolorization tank at a ratio of 1.2 Kg/ton of the dry basis and stirred at 110 rpm for 30 minutes for decolorization, and a plate and frame filter was used to filter and remove the activated carbon to obtain a decolorized sugar solution. The decolorization temperature for decolorization and filtration was controlled at 60° C., and the pH value for decolorization and filtration was in a range of 5.0-7.5.

Operation 5, fine filtration: a fine filtration operation was performed on the decolorized sugar solution obtained in operation 4 using a fine filtration membrane with an aperture of 0.45 μm to obtain a refined sugar solution. The temperature for fine filtration was controlled at 50° C.-65° C., and the pH value for fine filtration was in a range of 5.0-7.5.

Operation 6, evaporation and concentration: the refined sugar solution obtained in operation 5 was directed into an MVR evaporator for evaporation and concentration to obtain a concentrated sugar solution. The temperature for evaporation and concentration was at 68° C., and the pH value for evaporation and concentration was in a range of 5.0-7.5.

Operation 7, crystallization: the concentrated sugar solution obtained in operation 6 was introduced into a vacuum sugar-cooking system for crystallization. The temperature for crystallization was at 63° C.-65° C., and the vacuum level for crystallization was in a range of 70 mbar-90 mbar. Arabinose crystal seeds of 300-400 mesh were added in a ratio of two per ten thousand of the dry basis when an oversaturation degree of the concentrated sugar solution reached 1.01-1.02. A crystallization period was 8 hours, and a stirring speed was at 80 rpm.

Operation 8, centrifugation: a centrifugal separation operation was performed on a material obtained in operation 7 using a centrifuge machine, wherein a washing time was controlled at 10 seconds and a water temperature was maintained at 55° C.-60° C. A solid arabinose and an arabinose centrifugation mother liquor were separate from the material. The solid arabinose was dried in operation 9, the arabinose centrifugation mother liquor was directed through the pipeline into the blending tank for blending in operation 2, and the arabinose content in the arabinose centrifugation mother liquor was 89%.

Operation 9, drying and packaging: a drying operation was performed on the solid arabinose obtained in operation 8 with 80° C. hot air to obtain a high-purity arabinose crystal, wherein a moisture content was controlled in a range of 0.15%-0.3%, and after the moisture content was in the range of 0.15%-0.3%, the high-purity arabinose crystal was cooled to 20° C.-24° C. using clean cold air at 12° C.-15° C., and the arabinose content of the high-purity arabinose crystal is greater than 99.8%. The high-purity arabinose crystal obtained after drying was packaged using a packaging machine.

### Embodiment 3

Another embodiment of the method for preparing the high-purity arabinose crystal in the present disclosure includes:

Operation 1, dissolution: a purchased low-purity arabinose crystal was dissolved to obtain a dissolved sugar solution using a dissolution tank system. The dissolution tank system was provided with a temperature sensor and an automatic regulating valve, a Siemens DCS was used to monitor a temperature during the dissolution to ensure that a temperature of the dissolved sugar solution was in a range of 55° C.-60° C., and the arabinose content in the low-purity arabinose crystal was 97%.

Operation 2, blending: the dissolved sugar solution obtained in operation 1 was directed through a pipeline into a blending tank using an electromagnetic flowmeter, an automatic regulating valve, and a Siemens intelligent control system. An arabinose centrifugation mother liquor from operation 8 of a previous round of operations was added to the blending tank, and a blended sugar solution was obtained by blending the dissolved sugar solution with the arabinose centrifugation mother liquor. The pH value of the blended sugar solution was in a range of 4.3-5.0, a dry basis concentration was 60%, and the arabinose content of the blended sugar solution was 94.57%.

Operation 3, ion exchange: an ion exchange operation was performed on the blended sugar solution obtained in operation 2 to obtain an ion-exchanged sugar solution using an ion exchange system. The temperature of the ion-exchanged sugar solution was controlled at 45° C.-50° C. through a heat exchanger for ion exchange feed, a ratio of cationic resins to anionic resins of the ion exchange system was adjusted to be 7:10, and the pH value of the ion-exchanged sugar solution was 6.0.

Operation 4, decolorization and filtration: the ion-exchanged sugar solution was directed through a pipeline into a decolorization tank, activated carbon was added to the decolorization tank at a ratio of 1.4 Kg/ton of the dry basis and stirred at 110 rpm for 35 minutes for decolorization, and a plate and frame filter was used to filter and remove the activated carbon to obtain a decolorized sugar solution. The decolorization temperature for decolorization and filtration was controlled at 62° C., and the pH value for decolorization and filtration was in a range of 5.5-7.5.

Operation 5, fine filtration: a fine filtration operation was performed on the decolorized sugar solution obtained in operation 4 using a fine filtration membrane with an aperture of 0.45 μm to obtain a refined sugar solution. The temperature for fine filtration was controlled at 50° C.-65° C., and the pH value for fine filtration was in a range of 5.0-7.5.

Operation 6, evaporation and concentration: the refined sugar solution obtained in operation 5 was directed into an MVR evaporator for evaporation and concentration to obtain a concentrated sugar solution. The temperature for evaporation and concentration was at 68° C., and the pH value for evaporation and concentration was in a range of 5.0-7.5.

Operation 7, crystallization: the concentrated sugar solution obtained in operation 6 was introduced into a vacuum sugar-cooking system for crystallization. The temperature for crystallization was at 63° C.-65° C., and the vacuum level for crystallization was in a range of 70 mbar-90 mbar. Arabinose crystal seeds of 300-400 mesh were added in a ratio of two per ten thousand of the dry basis when an oversaturation degree of the concentrated sugar solution reached 1.01-1.02. A crystallization period was 8 hours, and a stirring speed was at 80 rpm.

Operation 8, centrifugation: a centrifugal separation operation was performed on a material obtained in operation 7 using a centrifuge machine, wherein a washing time was controlled at 10 seconds and a water temperature was maintained at 55° C.-60° C. A solid arabinose and an arabinose centrifugation mother liquor were separate from the material. The solid arabinose was dried in operation 9, the arabinose centrifugation mother liquor was directed through the pipeline into the blending tank for blending in operation 2, and the arabinose content in the arabinose centrifugation mother liquor was 91%.

Operation 9, drying and packaging: a drying operation was performed on the solid arabinose obtained in operation 8 with 80° C. hot air to obtain a high-purity arabinose crystal, wherein a moisture content was controlled in a range of 0.15%-0.3%, and after the moisture content was in the range of 0.15%-0.3%, the high-purity arabinose crystal was cooled to 20° C.-24° C. using clean cold air at 12° C.-15° C., and the arabinose content of the high-purity arabinose crystal is greater than 99.8%. The high-purity arabinose crystal obtained after drying was packaged using a packaging machine.

### Embodiment 4

Another embodiment of the method for preparing the high-purity arabinose crystal in the present disclosure includes:

Operation 1, dissolution: a purchased low-purity arabinose crystal was dissolved to obtain a dissolved sugar solution using a dissolution tank system. The dissolution tank system was provided with a temperature sensor and an automatic regulating valve, a Siemens DCS was used to monitor a temperature during the dissolution to ensure that a temperature of the dissolved sugar solution was in a range of 55° C.-60° C., and the arabinose content in the low-purity arabinose crystal was 96.5%.

Operation 2, blending: the dissolved sugar solution obtained in operation 1 was directed through a pipeline into a blending tank using an electromagnetic flowmeter, an automatic regulating valve, and a Siemens intelligent control system. An arabinose centrifugation mother liquor from operation 8 of a previous round of operations was added to the blending tank, and a blended sugar solution was obtained by blending the dissolved sugar solution with the arabinose centrifugation mother liquor. The pH value of the blended sugar solution was in a range of 4.3-5.0, a dry basis concentration was 55%, and the arabinose content of the blended sugar solution was 94.50%.

Operation 3, ion exchange: an ion exchange operation was performed on the blended sugar solution obtained in operation 2 to obtain an ion-exchanged sugar solution using an ion exchange system. The temperature of the ion-exchanged sugar solution was controlled at 45° C.-50° C. through a heat exchanger for ion exchange feed, a ratio of cationic resins to anionic resins of the ion exchange system was adjusted to be 7:10, and the pH value of the ion-exchanged sugar solution was 5.5.

Operation 4, decolorization and filtration: the ion-exchanged sugar solution was directed through a pipeline into a decolorization tank, activated carbon was added to the decolorization tank at a ratio of 1.25 Kg/ton of the dry basis and stirred at 110 rpm for 45 minutes for decolorization, and a plate and frame filter was used to filter and remove the activated carbon to obtain a decolorized sugar solution. The decolorization temperature for decolorization and filtration was controlled at 65° C., and the pH value for decolorization and filtration was in a range of 5.5-7.5.

Operation 5, fine filtration: a fine filtration operation was performed on the decolorized sugar solution obtained in operation 4 using a fine filtration membrane with an aperture of 0.45 μm to obtain a refined sugar solution. The temperature for fine filtration was controlled at 50° C.-65° C., and the pH value for fine filtration was in a range of 5.0-7.5.

Operation 6, evaporation and concentration: the refined sugar solution obtained in operation 5 was directed into an MVR evaporator for evaporation and concentration to obtain a concentrated sugar solution. The temperature for evaporation and concentration was at 70° C., and the pH value for evaporation and concentration was in a range of 5.0-7.5.

Operation 7, crystallization: the concentrated sugar solution obtained in operation 6 was introduced into a vacuum sugar-cooking system for crystallization. The temperature for crystallization was at 63° C.-65° C., and the vacuum level for crystallization was in a range of 70 mbar-90 mbar. Arabinose crystal seeds of 300-400 mesh were added in a ratio of two per ten thousand of the dry basis when an oversaturation degree of the concentrated sugar solution reached 1.01-1.02. A crystallization period was 8 hours, and a stirring speed was at 80 rpm.

Operation 8, centrifugation: a centrifugal separation operation was performed on a material obtained in operation 7 using a centrifuge machine, wherein a washing time was controlled at 10 seconds and a water temperature was maintained at 55° C.-60° C. A solid arabinose and an arabinose centrifugation mother liquor were separate from the material. The solid arabinose was dried in operation 9, the arabinose centrifugation mother liquor was directed through the pipeline into the blending tank for blending in operation 2, and the arabinose content in the arabinose centrifugation mother liquor was 90.3%.

Operation 9, drying and packaging: a drying operation was performed on the solid arabinose obtained in operation 8 with 80° C. hot air to obtain a high-purity arabinose crystal, wherein a moisture content was controlled in a range of 0.15%-0.3%, and after the moisture content was in the range of 0.15%-0.3%, the high-purity arabinose crystal was cooled to 20° C.-24° C. using clean cold air at 12° C.-15° C., and the arabinose content of the high-purity arabinose crystal is greater than 99.8%. The high-purity arabinose crystal obtained after drying was packaged using a packaging machine.

### Embodiment 5

Another embodiment of the method for preparing the high-purity arabinose crystal in the present disclosure includes:

Operation 1, dissolution: a purchased low-purity arabinose crystal was dissolved to obtain a dissolved sugar solution using a dissolution tank system. The dissolution tank system was provided with a temperature sensor and an automatic regulating valve, a Siemens DCS was used to monitor a temperature during the dissolution to ensure that a temperature of the dissolved sugar solution was in a range of 55° C.-60° C., and the arabinose content in the low-purity arabinose crystal was 96.9%.

Operation 2, blending: the dissolved sugar solution obtained in operation 1 was directed through a pipeline into a blending tank using an electromagnetic flowmeter, an automatic regulating valve, and a Siemens intelligent control system. An arabinose centrifugation mother liquor from operation 8 of a previous round of operations was added to the blending tank, and a blended sugar solution was obtained by blending the dissolved sugar solution with the arabinose centrifugation mother liquor. The pH value of the blended sugar solution was in a range of 4.3-5.0, a dry basis concentration was 60%, and the arabinose content of the blended sugar solution was 94.43%.

Operation 3, ion exchange: an ion exchange operation was performed on the blended sugar solution obtained in operation 2 to obtain an ion-exchanged sugar solution using an ion exchange system, wherein the temperature of the ion-exchanged sugar solution was controlled at 45° C.-50° C. through a heat exchanger for ion exchange feed, a ratio of cationic resins to anionic resins of the ion exchange system was adjusted to be 7:10, and the pH value of the ion-exchanged sugar solution was 6.2.

Operation 4, decolorization and filtration: the ion-exchanged sugar solution was directed through a pipeline into a decolorization tank, activated carbon was added to the decolorization tank at a ratio of 1.38 Kg/ton of the dry basis and stirred at 110 rpm for 40 minutes for decolorization, and a plate and frame filter was used to filter and remove the activated carbon to obtain a decolorized sugar solution. The decolorization temperature for decolorization and filtration was controlled at 60° C., and the pH value for decolorization and filtration was in a range of 5.5-7.5.

Operation 5, fine filtration: a fine filtration operation was performed on the decolorized sugar solution obtained in operation 4 using a fine filtration membrane with an aperture of 0.45 μm to obtain a refined sugar solution. The temperature for fine filtration was controlled at 50° C.-65° C., and the pH value for fine filtration was in a range of 5.0-7.5.

Operation 6, evaporation and concentration: the refined sugar solution obtained in operation 5 was directed into an MVR evaporator for evaporation and concentration to obtain a concentrated sugar solution. The temperature for evaporation and concentration was at 65° C., and the pH value for evaporation and concentration was in a range of 5.0-7.5.

Operation 7, crystallization: the concentrated sugar solution obtained in operation 6 was introduced into a vacuum sugar-cooking system for crystallization, wherein a temperature for crystallization was at 63° C.-65° C., and a vacuum level for crystallization was in a range of 70 mbar-90 mbar. Arabinose crystal seeds of 300-400 mesh were added in a ratio of two per ten thousand of the dry basis when an oversaturation degree of the concentrated sugar solution reached 1.01-1.02. A crystallization period was 8 hours, and a stirring speed was at 80 rpm.

Operation 8, centrifugation: a centrifugal separation operation was performed on a material obtained in operation 7 using a centrifuge machine, wherein a washing time was controlled at 10 seconds and a water temperature was maintained at 55° C.-60° C. A solid arabinose and an arabinose centrifugation mother liquor were separate from the material. The solid arabinose was dried in operation 9, the arabinose centrifugation mother liquor was directed through the pipeline into the blending tank for blending in operation 2, and the arabinose content in the arabinose centrifugation mother liquor was 89.8%.

Operation 9, drying and packaging: a drying operation was performed on the solid arabinose obtained in operation 8 with 80° C. hot air to obtain a high-purity arabinose crystal, wherein a moisture content was controlled in a range of 0.15%-0.3%, and after the moisture content was in the range of 0.15%-0.3%, the high-purity arabinose crystal was cooled to 20° C.-24° C. using clean cold air at 12° C.-15° C., and the arabinose content of the high-purity arabinose crystal is greater than 99.8%. The high-purity arabinose crystal obtained after drying was packaged using a packaging machine.

The technical effects of the method of preparing the high-purity arabinose crystal in the present disclosure are further illustrated below through a control group.

### Control Group

Conventional preparation operations for preparing an arabinose crystal were adopted for the control group, and a ratio of cationic resins to anionic resins was 1:1.

Operation 1, dissolution: a purchased low-purity arabinose crystal was dissolved to obtain a dissolved sugar solution using a dissolution tank system. The dissolution tank system was provided with a temperature sensor and an automatic regulating valve, a Siemens DCS was used to monitor a temperature during the dissolution to ensure that a temperature of the dissolved sugar solution was in a range of 55° C.-60° C., the arabinose content in the low-purity arabinose crystal was 97%, a dry basis concentration was 55%, and the pH value of the dissolved sugar solution was in a range of 3.5-5.0.

Operation 2, ion exchange: an ion exchange operation was performed on the dissolved sugar solution obtained in operation 2 to obtain an ion-exchanged sugar solution using an ion exchange system, wherein the temperature of the ion-exchanged sugar solution was controlled at 45° C.-50° C. through a heat exchanger for ion exchange feed, a ratio of cationic resins to anionic resins of the ion exchange system was adjusted to be 1:1, and the pH value of the ion-exchanged sugar solution was in a range of 3.5-4.0.

Operation 3, decolorization and filtration: the ion-exchanged sugar solution was directed through a pipeline into a decolorization tank, activated carbon was added to the decolorization tank at a ratio of 1.4 Kg/ton of the dry basis and stirred at 110 rpm for 45 minutes for decolorization, and a plate and frame filter was used to filter and remove the activated carbon to obtain a decolorized sugar solution. The decolorization temperature for decolorization and filtration was controlled at 75° C.-80° C., and a pH value for decolorization and filtration was in a range of 3.5-4.5.

Operation 4, fine filtration: a fine filtration operation was performed on the decolorized sugar solution obtained in operation 3 using a fine filtration membrane with an aperture of 0.45 μm to obtain a refined sugar solution. The temperature for fine filtration was controlled at 50° C.-65° C., and the pH value for fine filtration was in a range of 3.5-4.5.

Operation 5, evaporation and concentration: the refined sugar solution obtained in operation 4 was directed into a falling film evaporator for evaporation and concentration to obtain a concentrated sugar solution. The temperature for evaporation and concentration was in a arrange of 65° C.-98° C., and the pH value for evaporation and concentration was in a range of 3.5-4.5.

Operation 6, crystallization: the concentrated sugar solution obtained in operation 5 was introduced into a vacuum sugar-cooking system for crystallization. The temperature for crystallization was at 63° C.-65° C., and the vacuum level for crystallization was in a range of 70 mbar-90 mbar. Arabinose crystal seeds of 300-400 mesh were added in a ratio of two per ten thousand of the dry basis when an oversaturation degree of the concentrated sugar solution reached 1.01-1.02. A crystallization period was 8 hours, and a stirring speed was at 80 rpm.

Operation 7, centrifugation: a centrifugal separation operation was performed on a material obtained in operation 6 using a centrifuge machine, wherein a washing time was controlled at 10 seconds and a water temperature was maintained at 55° C.-60° C. A solid arabinose and an arabinose centrifugation mother liquor were separate from the material. The solid arabinose was dried in operation 8 and the arabinose centrifugation mother liquor was recycled back for use in dissolution in operation 1 through the pipeline.

Operation 8, drying and packaging: a drying operation was performed on the solid arabinose obtained in operation 7 with 80° C. hot air to obtain an arabinose crystal. The moisture content was controlled in a range of 0.15%-0.3%, and after the moisture content was in the range of 0.15%-0.3%, the arabinose crystal was cooled to 20° C.-24° C. using clean cold air at 12° C.-15° C. The arabinose crystal obtained after drying was packaged using a packaging machine.

TABLE 2
Comparison of key operational parameters and product yields
for the embodiments and the control group.

| | Operation | | | | | |
|---|---|---|---|---|---|---|
| | | De- | | | | |
| | | colori- | Evapor- | | | |
| | Ion | zation | ation | Arabinose | | |
| | ex- | temper- | temper- | content (%) | | Product |
| | change | ature | ature | | Final | yield |
| Group | pH | (° C.) | (° C.) | Blending | product | (%) |
| Embodiment | 6.5 | 60 | 68 | 93.84% | 99.85% | 96.83 |
| 2 | | | | | | |
| Embodiment | 6.0 | 62 | 68 | 94.57% | 99.82% | 96.32 |
| 3 | | | | | | |
| Embodiment | 5.5 | 65 | 70 | 94.50% | 99.81% | 96.98 |
| 4 | | | | | | |
| Embodiment | 6.2 | 60 | 65 | 94..43% | 99.92% | 96.19 |
| 5 | | | | | | |
| Control | 3.5~4.0 | 75~80 | 65~98 | / | 99.27 | 92.93 |
| group | | | | | | |

By referring to Table 2, the arabinose content in the arabinose crystal prepared in each embodiment is all above 99.8%, and the product yield exceeds 96%. The product yield refers to a ratio of the actual production of an arabinose crystal obtained to the theoretically calculated product yield. These results obtained according to the method of preparing the high-purity arabinose crystal in the present disclosure are superior to the arabinose crystal prepared in the control group, achieving the expected outcomes.

The above-described embodiments are merely the preferred embodiments of the present disclosure and should not be used to limit the scope of the present disclosure. Any modifications, equivalent substitutions, improvements, etc., made within the spirit and principles of the present disclosure should be included within the scope of protection of the present disclosure.

The basic concepts have been described above, and it is apparent that to a person skilled in the art, the above detailed disclosure is intended as an example only and does not constitute a limitation of the present disclosure. Although not expressly stated herein, various modifications, improvements, and amendments may be made to the present disclosure by those skilled in the art. Such modifications, improvements, and amendments are suggested in the present disclosure, so such modifications, improvements, and amendments remain within the spirit and scope of the exemplary embodiments of the present disclosure.

Also, the present disclosure uses specific words to describe the embodiments of the present disclosure. For example, “an embodiment,” “one embodiment,” and/or “some embodiments” are meant to refer to a certain feature, structure, or characteristic associated with at least one embodiment of the present disclosure. Accordingly, it should be emphasized and noted that “an embodiment” or “one embodiment” or “an alternative embodiment” mentioned two or more times in different places in the present disclosure do not necessarily refer to the same embodiment. Furthermore, certain features, structures, or characteristics in one or more embodiments of the present disclosure may be suitably combined.

In addition, unless expressly stated in the claims, the order of processing elements and sequences, the use of numerical letters, or the use of other names described herein are not intended to limit the order of the processes and methods of the present disclosure. Although a number of embodiments of the present disclosure currently considered useful are discussed in the above disclosure by way of various examples, it should be understood that such details serve illustrative purposes only, and that additional claims are not limited to the disclosed embodiments; rather, the claims are intended to cover all amendments and equivalent combinations that are consistent with the substance and scope of the embodiments of the present disclosure. For example, although the implementation of various components described above may be embodied in a hardware device, it may also be implemented as a software only solution, e.g., an installation on an existing server or mobile device.

Similarly, it should be noted that in order to simplify the presentation of the present disclosure, and thus aid in the understanding of one or more embodiments of the present disclosure, the preceding description of embodiments of the present disclosure sometimes combines a plurality of features into a single embodiment, accompanying drawings, or description thereof. However, this way of disclosure does not imply that the subject matter of the present disclosure requires more features than those mentioned in the claims. Rather, claimed subject matter may lie in less than all features of a single foregoing disclosed embodiment.

In some embodiments, numeric values describing the composition and quantity of attributes are used in the description. It should be understood that such numeric values used for describing embodiments may be modified with qualifying terms such as “about,” “approximately,” or “generally.” Unless otherwise stated, “about,” “approximately,” or “generally” indicates that a variation of +20% is permitted in the described numbers. Accordingly, in some embodiments, the numerical parameters used in the disclosure and claims are approximations, which may change depending on the desired characteristics of the individual embodiment. In some embodiments, the numerical parameters should take into account a specified number of valid digits and employ a general manner of bit retention. Although the numerical ranges and parameters used in some embodiments of the present disclosure to confirm the breadth of the range are approximations, in specific embodiments, such numerical values are set as precisely as practicable.

With respect to each of the patents, patent applications, publications of patent applications, and other material, such as articles, books, specifications, publications, documents and the like, cited in the present disclosure, the entire contents thereof are hereby incorporated herein by reference. Application history documents that are inconsistent with the contents of the present disclosure or that create conflicts are excluded, as are documents (currently or hereafter appended to the present disclosure) that limit the broadest scope of the claims of the present disclosure. It should be noted that in the event of any inconsistency or conflict between the descriptions, definitions, and/or use of terminology in the materials appended to the present disclosure and the contents described herein, the descriptions, definitions, and/or use of terminology in the present disclosure shall prevail.

Finally, it should be understood that the embodiments described in the present disclosure are used only to illustrate the principles of the embodiments of the present disclosure. Other deformations may also fall within the scope of the present disclosure. Therefore, by way of example and not limitation, alternative configurations of the embodiments disclosed in the present disclosure may be considered consistent with the teachings of the present disclosure. Accordingly, the embodiments described in the present disclosure are not limited to the explicitly introduced and described embodiments in the present disclosure.

What is claimed is:

1. A method for preparing a high-purity arabinose crystal, comprising:
operation 1, dissolution: dissolving a low-purity arabinose crystal to obtain a dissolved sugar solution using a dissolution tank system, wherein the dissolution tank system is provided with a temperature sensor and an automatic regulating valve, a Siemens distributed control system (DCS) is used to monitor a temperature during the dissolution to ensure that a temperature of the dissolved sugar solution is in a range of 55° C.-60° C., and an arabinose content in the low-purity arabinose crystal is 96%-97%;
operation 2, blending: directing the dissolved sugar solution through a pipeline into a blending tank, adding an arabinose centrifugation mother liquor from operation 8 of a previous round of operations to the blending tank, and obtaining a blended sugar solution by blending the dissolved sugar solution with the arabinose centrifugation mother liquor, wherein a pH value of the blended sugar solution is in a range of 4.3-5.0, a dry basis concentration is in a range of 50%-60%, and the arabinose content of the blended sugar solution is 94±0.5%;
operation 3, ion exchange: performing an ion exchange operation on the blended sugar solution to obtain an ion-exchanged sugar solution, wherein a temperature of the ion-exchanged sugar solution is controlled at 45° C.-50° C. through a heat exchanger for ion exchange feed, and a pH value of the ion-exchanged sugar solution is in a range of 5.5-6.5;
operation 4, decolorization and filtration: directing the ion-exchanged sugar solution through a pipeline into a decolorization tank for decolorization and filtration to obtain a decolorized sugar solution, wherein a decolorization temperature for decolorization and filtration is controlled at 60° C.-65° C., and a pH value for decolorization and filtration is in a range of 5.5-7.5;
operation 5, fine filtration: performing a fine filtration operation on the decolorized sugar solution using a fine filtration membrane with an aperture of 0.45 μm to obtain a refined sugar solution, wherein a temperature for fine filtration is controlled at 50° C.-65° C., and a pH value for fine filtration is in a range of 5.0-7.5;
operation 6, evaporation and concentration: directing the refined sugar solution into a mechanical vapor recompression (MVR) evaporator for evaporation and concentration to obtain a concentrated sugar solution, wherein a temperature for evaporation and concentration is at 65° C.-70° C., and a pH value for evaporation and concentration is in a range of 5.0-7.5;
operation 7, crystallization: introducing the concentrated sugar solution into a vacuum sugar-cooking system for crystallization, wherein a temperature for crystallization is at 63-65° C., and a vacuum level for crystallization is in a range of 70 mbar-90 mbar;
operation 8, centrifugation: performing a centrifugal separation operation on a material obtained in operation 7 to separate a solid arabinose and an arabinose centrifugation mother liquor from the material, wherein the arabinose centrifugation mother liquor is directed through the pipeline into the blending tank for blending in operation 2 and an arabinose content in the arabinose centrifugation mother liquor is in a range of 89%-91%.
operation 9, drying: performing a drying operation on the solid arabinose with 80° C. hot air to obtain the high-purity arabinose crystal, wherein an arabinose content is greater than 99.8%.

2. The method of claim 1, wherein in operation 3, an ion exchange column is used in the ion exchange operation, and a ratio of cationic resins to anionic resins in the ion exchange column is 7:10.

3. The method of claim 1, wherein in operation 4, the decolorization and filtration operation includes adding activated carbon to the decolorization tank at a ratio of 1.2-1.4 Kg/ton of the dry basis, stirring at 110 rpm for 30-45 minutes for decolorization, and using a plate and frame filter to filter and remove the activated carbon.

4. The method of claim 1, wherein in operation 7, the crystallization operation includes performing a vacuum evaporation crystallization by adding arabinose crystal seeds of 300-400 mesh in a ratio of two per ten thousand of the dry basis when an oversaturation degree of the concentrated sugar solution reaches 1.01-1.02, a crystallization period is 8 hours, and a stirring speed is at 80 rpm.

5. The method of claim 1, wherein in operation 8, the centrifugal separation operation further includes controlling a washing time at 10 seconds and maintaining a water temperature at 55° C.-60° C.

6. The method of claim 1, wherein in operation 9, the drying operation further includes controlling a moisture content in a range of 0.15%-0.3%, and after the moisture content is in the range of 0.15%-0.3%, cooling the high-purity arabinose crystal to 20° C.-24° C. using clean cold air with a temperature in a range of 12° C.-15° C.
